(19) United States
(12) Patent Application Publication
(10) Pub. No.: US 2016/0000214 A1
(43) Pub. Date: Jan. 7, 2016

(54) ELECTRIC TOOTHBRUSH, TOOTHBRUSH BRISTLE ASSEMBLY, AND METHOD FOR MANUFACTURING THE TOOTHBRUSH BRISTLE ASSEMBLY

(71) Applicant: Young Ha KIM, Choongcheongnam-do (KR)

(72) Inventor: Young Ha KIM, Choongcheongnam-do (KR)

(21) Appl. No.: 14/855,575

(22) Filed: Sep. 16, 2015

Related U.S. Application Data
(62) Division of application No. 13/812,031, filed on Jan. 24, 2013.

(30) Foreign Application Priority Data
Jul. 30, 2010 (KR) 1020100074073
Jul. 7, 2011 (KR) 1020110067311

(51) Int. Cl. A46B 15/00; A61N 5/06; A61C 19/06; A61C 17/22

(52) U.S. Cl. CPC A46B 15/0034; A61C 17/222; A61N 5/06; A61C 19/066; A61N 2005/0651

(57) ABSTRACT

One aspect of the present invention relates to an electric toothbrush in which an LED unit is mounted to perform oral sterilization and tooth whitening treatment during the brushing of the teeth, and further relates to a toothbrush bristle assembly for the electric toothbrush. The electric toothbrush according to one embodiment of the present invention comprises: a toothbrush bristle assembly including a handle having a vibrator, and a coupling space into which the vibrator is coupled. The toothbrush bristle assembly comprises: a toothbrush bristle body having a brush; an LED unit mounted on the toothbrush bristle body to perform oral sterilization and tooth whitening treatment; and a connector module having a first connector arranged in the toothbrush bristle body to interconnect the LED unit and a driving power source, as well as a second connector arranged in the vibrator.

### CROSS-REFERENCE TO RELATED APPLICATION

This application claims the benefit of Korean Patent Application No. 10-2010-0074073, filed on Jul. 30, 2010 and Korean Patent Application No. 10-2011-0067311, filed on Jul. 7, 2011 in the Korean Intellectual Property Office, the disclosure of which is incorporated herein by reference.

### FIELD OF THE INVENTION

The present invention relates to an electric toothbrush and a method for manufacturing it, more specifically, an electric toothbrush and a toothbrush bristle assembly thereof which can perform oral sterilization and tooth whitening treatment during the brushing of the teeth by mounting a LED unit.

### DESCRIPTION OF RELATED ART

In general, people eat foods for their activities and the eaten foods are mixed with spit through chewing process in the mouth and then transferred to digestion organs. The foods supplied into the mouth are finely pulverized by arrays of teeth arranged opposite each other abutting against each other according to upward and downward movement of jaws. During such a process, the fragments of the foods are necessarily caught in gaps between teeth or between teeth and gums.

If the foods caught in the gaps as described above are not removed timely, the foods are decomposed and decayed by germs in the mouth and cause various oral disease, periodontal disease or foul breath etc., and therefore, for preventing it, a toothbrush for brushing the teeth has been devised.

Particularly, in recent, by embedding LED into the toothbrush and using the wavelength of LED light, the germs in the mouth are sterilized and disinfection and tooth whitening treatment are performed during the brushing of the teeth.

However, there are problems that, in a process of thus embedding the LED into the toothbrush, the toothbrush is unnecessarily structured and becomes complex or the LED is not stably driven.

Furthermore, there is also a problem that, in the process of embedding the LED into the toothbrush, waterproof of the toothbrush is not made, thus the toothbrush may be easily failed.

### SUMMARY OF THE PRESENT INVENTION

An aspect of the present invention aims at providing an electric toothbrush and a toothbrush bristle assembly thereof which can perform oral sterilization and tooth whitening treatment during the brushing of the teeth by mounting a LED unit.

Another aspect of the present invention aims at providing an electric toothbrush and a toothbrush bristle assembly thereof wherein stable driving control of the LED unit can be achieved through a connector module interconnecting a driving power source and the LED unit.

Yet another aspect of the present invention aims at providing an electric toothbrush and a toothbrush bristle assembly thereof which can prevent internal electric components of the electric toothbrush from being damaged by penetrating water by ensuring waterproof function when mounting the LED unit and the connector modules in a toothbrush bristle body.

An electric toothbrush according to an example of the present invention is characterized in that it comprises a handle having a vibrator and a toothbrush bristle assembly having a coupling space coupled with the vibrator and the toothbrush bristle assembly comprises a toothbrush bristle body provided with a brush; a LED unit mounted on the toothbrush bristle body for performing oral sterilization and tooth whitening treatment; and a connector module having a first connector arranged within the toothbrush bristle body for interconnecting the LED unit and a driving power source and a second connector arranged within the vibrator.

Furthermore, the present invention is characterized in that the first connector comprises at least two strands of first connector pins; a first connector socket formed at one end portion of the first connector pins; and a first connector cover inserted and installed from the other end portion of the first connector pins and the second connector comprises at least two strands of pins inserted in the first connector socket.

Furthermore, the present invention is characterized in that the first connector comprises a plug-type connector mounted in series with a substrate for LED light source by at least two strands of pins and the second connector comprises a plastic material-made body part defining the exterior and at least two strands of pin parts formed long within the body part and inserted into the plug-type connector.

Furthermore, the present invention is characterized in that the toothbrush bristle body comprises a first toothbrush bristle body and a second toothbrush bristle body which are formed by a double injection molding method.

Furthermore, the present invention is characterized in that the toothbrush bristle assembly comprises a first toothbrush bristle body formed of synthetic resin by a first injection molding so that a first accommodation space is formed; LED unit and first connector having a waterproof portion which has to avoid a contact with water or moisture and accommodated and joined in the first accommodation space of the first toothbrush bristle body; and a second toothbrush bristle body formed by a second injection molding to be joined to the first toothbrush bristle body while accommodating the LED unit and the first connector in a second accommodation space so as to enclose the waterproof portion of the LED unit and first connector.

Furthermore, the present invention is characterized in that the LED unit comprises LED light sources mounted on the toothbrush bristle body for irradiating LED light; and a substrate for LED light source which is provided within the toothbrush bristle body by the double injection molding of the toothbrush bristle body and controls the driving of LED light sources.

Furthermore, the present invention is characterized in that the LED light sources comprise red and white LED light sources.

Furthermore, the present invention is characterized in that the coupling space of the toothbrush bristle body and the vibrator inserted into the coupling space of the toothbrush bristle body are provided in the form of dome.

Furthermore, the present invention is characterized in that the second connector has its outer circumferential surface formed corresponding to an inner circumferential surface of the vibrator so that the pins can be fixed within the vibrator without a shake.

A toothbrush bristle assembly according to an example of the present invention is characterized in that it comprises a toothbrush bristle body having a first toothbrush bristle body and a second toothbrush bristle body which are formed by a double injection molding method; a LED unit comprising LED light sources for irradiating LED light to perform oral sterilization and tooth whitening treatment, and a substrate for LED light source mounted between the first toothbrush bristle body and the second toothbrush bristle body to control the driving of LED light sources; and a connector module having a first connector arranged within the toothbrush bristle body for interconnecting the LED unit and a driving power source.

A method for manufacturing a toothbrush bristle assembly according to an example of the present invention is characterized in that the method comprises a step of forming a first toothbrush bristle body of synthetic resin by a first injection molding so that a first accommodation space is formed; a step of assembling a LED unit and a first connector having a waterproof portion so that the unit and the first connector are accommodated and joined in the first accommodation space of the first toothbrush bristle body; and a step of forming a second toothbrush bristle body by a second injection molding to be joined to the first toothbrush bristle body while accommodating the LED unit and the first connector in a second accommodation space.

Therefore, the electric toothbrush and toothbrush bristle assembly thereof according to an example of the present invention have an effect that they can perform oral sterilization and tooth whitening treatment during the brushing of the teeth by mounting a LED unit.

Furthermore, the electric toothbrush and toothbrush bristle assembly thereof according to an example of the present invention have an effect that stable driving control of the LED unit can be achieved through a connector module interconnecting a driving power source and the LED unit.

Furthermore, the electric toothbrush and toothbrush bristle assembly thereof according to an example of the present invention have an effect that they can prevent internal electric components of the electric toothbrush from being damaged by penetrating water by ensuring waterproof function when mounting the LED unit and the connector modules in a toothbrush bristle body.

### BRIEF DESCRIPTION OF THE DRAWINGS

FIG. 1 is a front view showing an electric toothbrush according to one example of the present invention.

FIG. 2 is a side sectional view showing the electric toothbrush according to the one example of the present invention.

FIG. 3 is a view showing a disassembled state of a toothbrush bristle assembly and a handle of the electric toothbrush according to the one example of the present invention.

FIG. 4 is a sectional view showing the toothbrush bristle assembly of the electric toothbrush according to the one example of the present invention.

FIG. 5 is a view showing a first toothbrush bristle body and first connector of a toothbrush bristle assembly of the electric toothbrush according to the one example of the present invention.

FIG. 6 is a sectional view showing an electric toothbrush according to another example of the present invention.

FIG. 7 is a perspective view showing the electric toothbrush according to the another example of the present invention.

FIG. 8 is an enlarged view showing a toothbrush bristle assembly of the electric toothbrush illustrated in FIG. 6.

<list of reference numbers>

10: electric toothbrush 100: handle
110: vibrator 111: cylindrical part
112: shaft part 120: support member
130: battery 140: driving circuit substrate
150: electric motor 160: weight
200: toothbrush bristle assembly 210: toothbrush bristle body
220: first toothbrush bristle body 230: second toothbrush bristle body
300: LED unit 310: LED light source
320: substrate for LED light source 400: first connector
410: first connector pin 420: first connector socket
430: first connector cover 500: second connector
510: electrode pin 520: pin support

### MODE FOR CARRYING OUT THE INVENTION

In the following, preferred examples of the present invention will be described in detail with reference to the attached drawings.

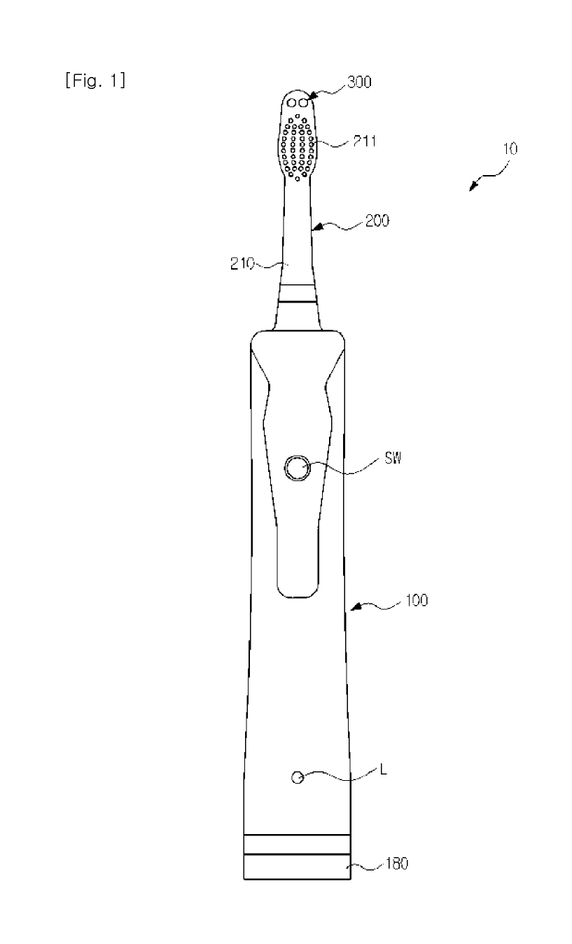

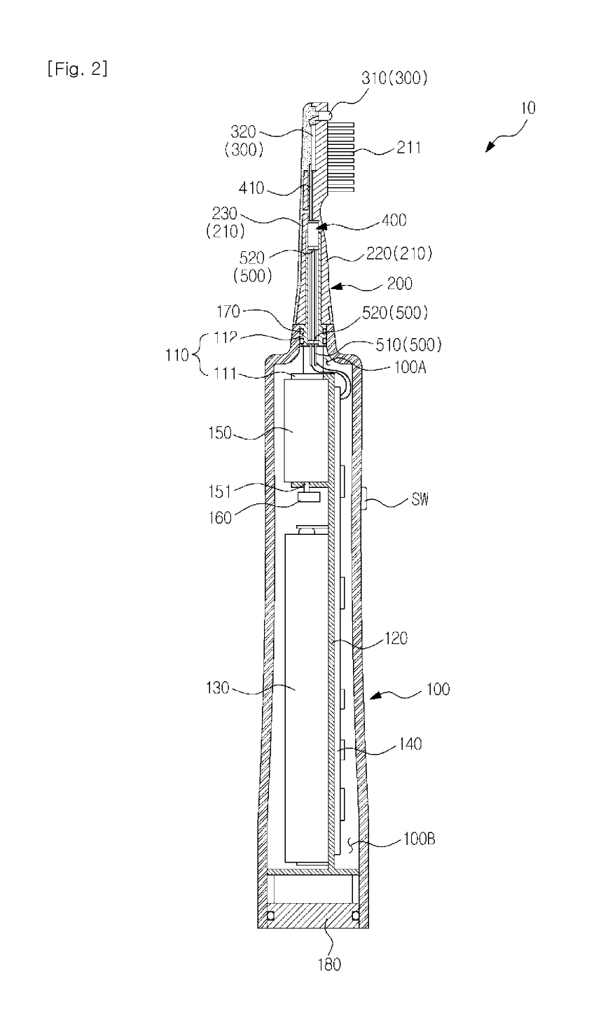

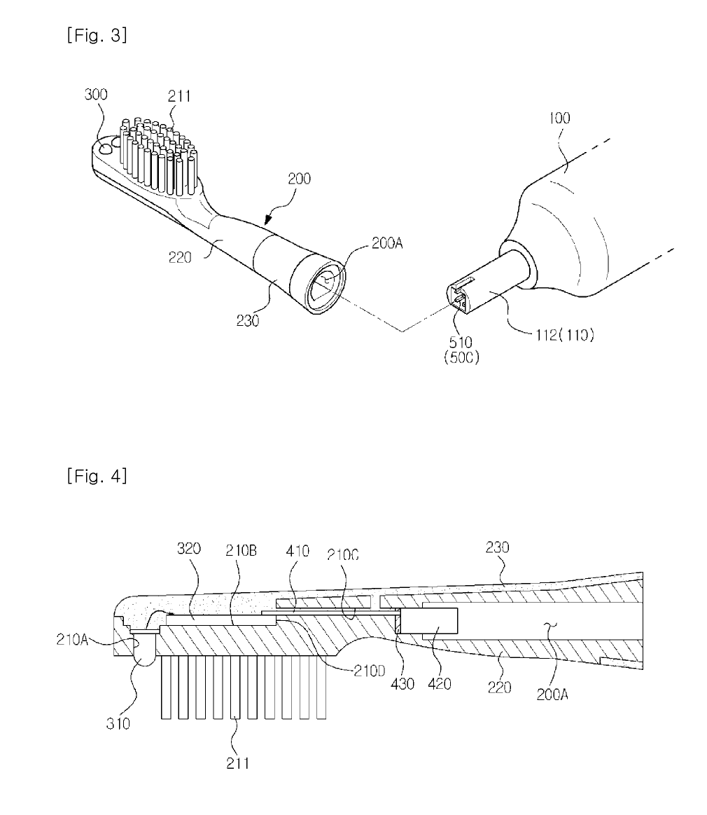

FIG. 1 is a front view showing an electric toothbrush according to one example of the present invention, FIG. 2 is a sectional view showing the electric toothbrush according to the one example of the present invention, and FIG. 3 is a view showing a disassembled state of a toothbrush bristle assembly and a handle of the electric toothbrush according to the one example of the present invention.

As illustrated in FIGS. 1 to 3, the electric toothbrush (10) according to the one example of the present invention comprises a handle (100) having a vibrator (110), a toothbrush bristle assembly (200) coupled with the vibrator (110), an LED unit (300) of the toothbrush bristle assembly (200) for performing oral sterilization and tooth whitening treatment during the brushing of the teeth, and connector modules (400, 500) for electrically interconnect a driving power source (130) and the LED unit (300).

The handle (100) is extendedly formed with a certain length for enabling a user to operate the handle. The handle (100) is in the form of a tube as a whole and has a first opening (100A) and a second opening (100B) at a front end portion and a rear end portion of the handle, respectively.

A support member (120) for supporting internal electric components is arranged within the handle (100). This support member (120) can be detachably joined within the handle through the first opening (100A) and the second opening (100B) of the handle.

Arranged on the support member (120) are a battery (130) as driving power source and a driving circuit substrate (140) for controlling the driving of an electric motor (150) (or the vibrator (110)) as will be described later.

Also, the vibrator (110) is arranged at a front end portion of the support member (120). The vibrator (110) comprises a cylindrical part (111) formed integrally with the support member (120) at the front end portion of the support member (120) and a shaft part (112) formed integrally with the cylindrical part (111) and protruding out of the handle (100) while closing the first opening (100A).

The vibrator (110) is inserted in a coupling space (200A) of the toothbrush bristle assembly (200) described later and delivers vibration to the toothbrush bristle assembly (200) while contacting with the toothbrush bristle assembly (200).

The cylindrical part (111) of the vibrator (110) includes the electric motor (150) as vibrating source and a weight (160) fixed to a rotation shaft (151) of the electric motor (150). The weight (160) is configured to have its center offset from a rotational center of the rotation shaft (151) of the electric motor. For such a vibrator (110), if a switch is ON, the weight (160) is rotated by the electric motor (150) and the toothbrush bristle assembly (200) is vibrated about the vibrator (110) by centrifugal force of the weight (160).

Arranged within the shaft part (112) of the vibrator (110) are the connector modules (400, 500) connected with a driving circuit substrate (140) by conductive lines, detailed description of which will be given later.

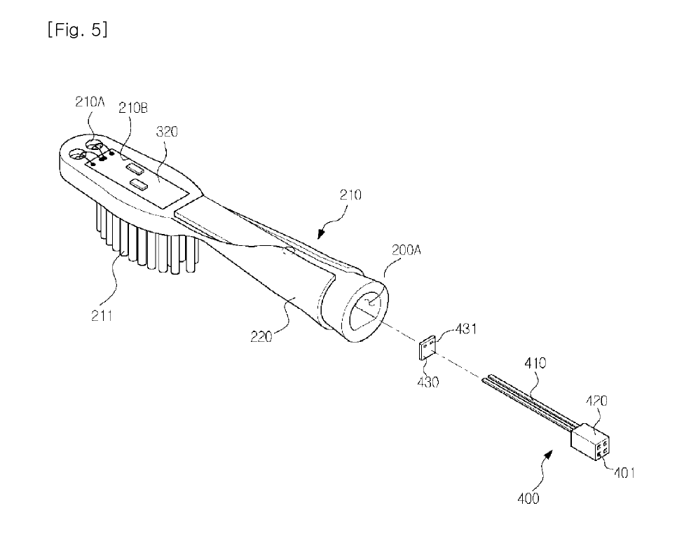

FIG. 4 is a sectional view showing the toothbrush bristle assembly of the electric toothbrush according to the one example of the present invention, and FIG. 5 is a view showing a first toothbrush bristle body and first connector of the toothbrush bristle assembly of the electric toothbrush according to the one example of the present invention.

As illustrated in FIGS. 2 to 5, the toothbrush bristle assembly (200) is mounted so as to cover the shaft part of the vibrator, wherein such a toothbrush bristle assembly (200) comprises a toothbrush bristle body (210) provided with a brush, the LED unit (300), the first connector (400) of the connector modules (400, 500).

The toothbrush bristle body (210) is a consumable part and is configured so as to be removable from the handle (100) so that the body can be replaced, wherein the brush (211) for washing the teeth is arranged at a front end portion of such a toothbrush bristle body (210). Here, the toothbrush bristle body (210) according to the example of the present invention comprises a first toothbrush bristle body (220) and a second toothbrush bristle body (230) which are formed by a double injection molding method, which double injection molding method will be described later.

The toothbrush bristle body (210) is equipped with the LED unit (300) for performing the tooth whitening treatment and oral sterilization by irradiating the LED light in the mouth.

Such a LED unit (300) comprises LED light sources (310) mounted on the toothbrush bristle body (210) for irradiating LED light, and a substrate (320) for LED light source which is provided within the toothbrush bristle body (210) by the double injection molding of the toothbrush bristle body (210) and controls the driving of LED light sources (310).

The LED light sources (310) are formed so as to extend out of the toothbrush bristle body (210) from the inside of the body at the front end portion thereof.

The LED light sources (310) are mounted in first accommodation spaces (210A), wherein such LED light sources (310) are disposed at a constant interval in a longitudinal direction and may be provided in a quantity of at least two.

The LED light sources (310) thus disposed in a quantity of at least two may include LED light sources (310) having different bands of wavelength. As such, two types of LED light sources (310) having different bands of wavelength are each arranged in the first A accommodation spaces (210A) to irradiate the LED light having different bands of wavelength in the user's mouth, thereby exhibiting different effect from band to band, whereby the user can obtain various effects at the same time. For example, the LED light sources (310) may consist of a red LED light source (310) and a white LED light source (310).

Provided in the toothbrush bristle body (210) is the first connector (400) of the connector modules (400, 500) for supplying power to the substrate (320) for LED light source so as to selectively operate the LED light sources (310).

Such a first connector (400) comprises first connector pins (410) connected to the substrate (320) for LED light source, a first connector socket (420) formed at one end portion of the first connector pins (410) and supplied with electric power from the battery, and a first connector cover (430) inserted and installed from the other end portion of the first connector pins (410).

The first connector pins (410) are two strands of continuously-formed pins which have the same plane, and are connected to the substrate (320) for LED light source.

The first connector socket (420) arranged at one end portion of the first connector pins (410) is in the form of rectangular parallelepiped and has at least two socket grooves (421). Electrode pins (510) of the second connector (500) connected to the driving circuit substrate (140) are inserted into these socket grooves (421) and supplied with driving electric power from the battery (130).

The first connector cover (430) has at least two through holes (431) into which the other end portions of the first connector pins (410) is inserted. Such a first connector cover (430) is formed corresponding to inner circumferential surface of the coupling space (200) of the toothbrush bristle body (210).

For example, if the coupling space (200A) of the toothbrush bristle body (210) is in the form of dome, the first connector cover (430) may be in the form of a rectangular plate which can be elastically deformed so that the cover is tightly fitted in the toothbrush bristle body (210). It is for a purpose that the first connector (400) can be tightly joined in the toothbrush bristle body (210) without a play relative thereto in a process of assembling the first connector cover (430) with a front end portion of the coupling space of the toothbrush bristle body.

Meanwhile, if the coupling space (200A) of the toothbrush bristle body (210) is in the form of dome and the shaft part (112) of the vibrator (110) inserted into the coupling space (200A) of the toothbrush bristle body (210) is also in the form of dome, an advantage is obtained that a relative rotation of the toothbrush bristle body (210) and the vibrator (110)(or handle (100)) is inhibited.

Provided within the shaft part (112) of the vibrator (110) is the second connector (500) of the connector modules (400, 500) connected to the driving circuit substrate (140) by conductive lines.

The second connector (500) may comprise the electrode pins (510) connected to the driving circuit substrate (140) by conductive lines within the shaft part (112) of the vibrator (110), and a pin support (520) for supporting both end portions of the electrode pins (510).

The electrode pin (510) is a continuous linear pin, wherein one end portion of the pin is connected to the driving circuit substrate (140) by conductive lines and the other end portion is inserted into the socket groove (421) of the first connector (400).

The pin support (520) has its outer circumferential surface formed corresponding to an inner circumferential surface of the shaft part (112) so that the electrode pin (510) can be fixed within the shaft part (112) of the vibrator (110) without a shake.

Therefore, by delivering a signal produced in the driving circuit substrate (140) by the driving power source (130) to the substrate (320) for LED light source via the electrode pin (510) of the second connector (500) and then the first connector pin (410) of the first connector (400), the LED light source (310) selectively emits the light, thereby performing oral sterilization and tooth whitening treatment during the brushing of the teeth.

Meanwhile, the battery (130) and the driving circuit substrate (140) are electrically connected to each other and the driving circuit substrate (140) and the electric motor (150) are also electrically connected to each other by conductive lines (not illustrated).

Next, with reference to FIGS. 4 and 5, a description will be given of a double injection molding method of the toothbrush bristle body according to an example of the present invention.

The double injection molding method performs a forming by filling a second resin into a space between a first formed product initially formed and a second cavity by means of a metal die designed to perform a double injection utilizing two injection devices and injector or core of structure wherein a rotating mechanism is installed on a movable plate or a slide structure, using two types of different resins or resins of two different colors, wherein since the first cavity and the second cavity are accurately replaced, limitation on a shape is less and applicability of product is wide, thus diversification of design is possible. Furthermore, since the double injection molding method performs an injection at a time, it can produce a product by a single forming step, contrary to an existing normal injection molding method wherein, for producing a product, two parts are formed through two steps and a secondary process such as fusion or painting process is then performed, thereby reducing the prime cost and allowing production of products of various designs. For the toothbrush bristle body according to the example of the present invention, a first toothbrush bristle body corresponds to the first formed product and a second toothbrush bristle body is formed by the second forming step.

The first toothbrush bristle body (220) is firstly formed of synthetic resin so that the first accommodation space (210A-210C) is formed. The first accommodation space (210A-210C) of the first toothbrush bristle body (220) comprises the first A accommodation space (210A) in which the LED light source (310) is inserted, the first B accommodating space (210B) in which the substrate (320) for LED light source electrically connected to the LED light source (310) is joined, and the first C accommodation space (210C) in which a first connector (400) electrically connected to the substrate (320) for LED light source is joined.

The first A accommodation space (210A) is formed so as to extend from the inside of the front end portion of the first toothbrush bristle body (220) to the outside thereof for insertion of the LED light source (310). Such first A accommodation spaces (210A) are formed at the front end portion of the first toothbrush bristle body (220) in a quantity of two and red and white LED light sources (310) can be each inserted in the first A accommodation spaces.

Furthermore, formed adjacent to the first A accommodation space (210A) is the first B accommodating space (210B) in which the substrate (320) for LED light source is joined. The first B accommodation space (210B) is formed with a continuously flat surface so that one end portion of the space is adjacent to the first A accommodation space (210A) while the other end portion is formed with a step (210D) having a height corresponding to the substrate (320) for LED light source. Therefore, if the substrate (320) for LED light source is joined in the first B accommodation space (210B), the substrate (320) for LED light source can have the same height without any gap relative to the first toothbrush bristle body (220).

Furthermore, formed adjacent to the first B accommodation space (210B) is the first C accommodation space (210C) consisting of a gap for joining the first connector (400) electrically connected to the substrate (320) for LED light source. The first C accommodation space (210C) is a space formed within the first toothbrush bristle body (220), in which the first connector pins (410) of the first connector (400) are disposed so as to be electrically connected to the substrate (320) for LED light source.

That is to say, the LED unit (300) and first connector (400) described above have a water-proof portion which has to avoid a contact with water or moisture and are accommodated and joined in the first A˜C accommodation spaces (210A˜210C) of the first toothbrush bristle body (220).

Meanwhile, at its rear end portion, the first toothbrush bristle body (220) has the coupling space (200A) for removable coupling of the handle (100).

Also, the second toothbrush bristle body (230) is joined to the first toothbrush bristle body (220) while accommodating the LED unit (300) and the first connector (400) in an accommodation space (not illustrated) so as to enclose the water-proof portion of the LED unit (300) and the first connector (400).

That is to say, the toothbrush bristle body (210) illustrated in FIG. 4 is formed by the double injection molding method wherein the first accommodation spaces (210A˜210C) and coupling space (200A) of the first toothbrush bristle body (220) are formed at the time of the first injection molding, as shown in FIG. 5, and the second toothbrush bristle body (230) is formed at the time of the second injection molding in a manner that the first accommodation spaces (210A˜210C) or gap of the first toothbrush bristle body (220) is blocked.

Therefore, the second accommodation space of the second toothbrush bristle body (230) accommodates and joins the LED unit (300) and the first connector (400), thereby blocking each gap of the first toothbrush bristle body (220), and since a joining strength of the first toothbrush bristle body (220) and the second toothbrush bristle body (230) is enhanced, the water cannot penetrate into a joined portion of the first toothbrush bristle body (220) and the second toothbrush bristle body (230), whereby a water-proof function is provided.

Meanwhile, the first connector cover (430) of the first connector (400) is joined to the front end portion of the coupling space (200A) of the first toothbrush bristle body (220) in close contact with the front end portion to thereby prevent injected material from flowing out toward a rear end portion of the first connector socket (420) or the coupling space of the first toothbrush bristle body (220) at the time of the second injection molding, thus can prevent electrical defects of the first connector (400) or inferior quality of the first toothbrush bristle body (220).

Furthermore, an O-ring (170) is further joined to an outer circumferential surface of the shaft part (112) of the vibrator (110) to prevent the water from penetrating into between the handle (100) and the toothbrush bristle assembly (200). Also, SW (not illustrated) designates a switch, L designates a filling indication lamp, reference number “180” designates a handle cover for closing the second opening.

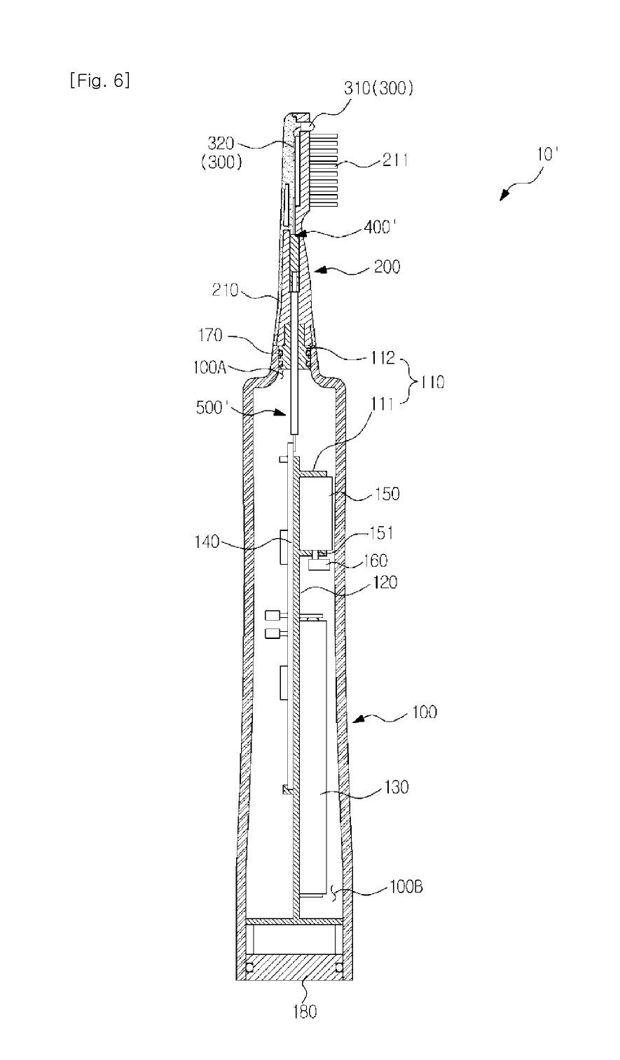

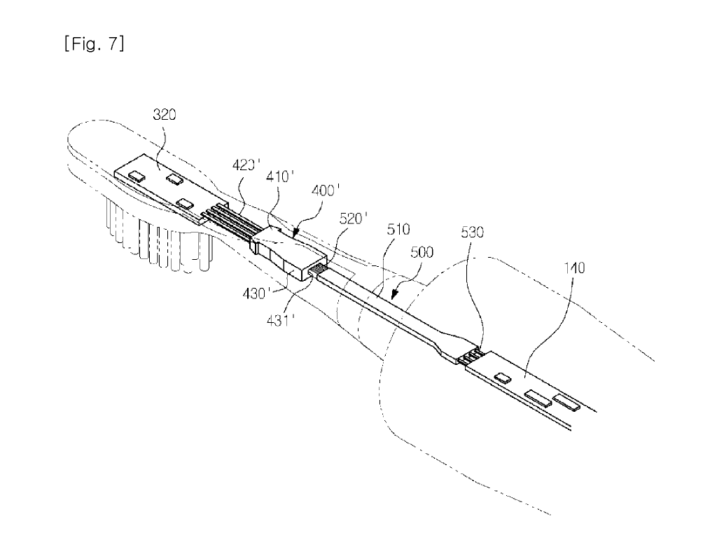

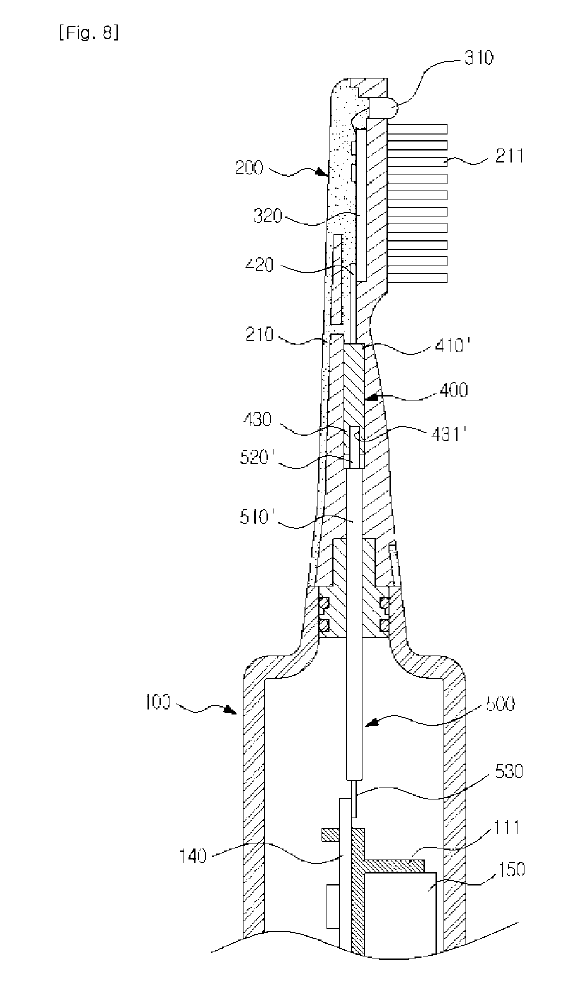

Another example of the present invention will be described with reference to FIGS. 6 to 8. The same components as those of the example described previously are designated by the same reference numbers, and description thereof will be omitted. FIG. 6 is a sectional view showing an electric toothbrush according to the another example of the present invention, FIG. 7 is a perspective view showing the electric toothbrush according to the another example of the present invention, and FIG. 8 is an enlarged view showing a toothbrush bristle assembly of the electric toothbrush illustrated in FIG. 6.

As illustrated in FIGS. 6 to 8, the electric toothbrush (10′) according to the another example of the present invention comprises a handle (100) having a vibrator (110), a battery (130) arranged in the handle (100), a toothbrush bristle assembly (200) coupled with the vibrator (110), an LED unit (300) of the toothbrush bristle assembly (200) for performing the oral sterilization and tooth whitening treatment during the brushing of the teeth, and connector modules (400′, 500′) for connecting the driving power source (130) with the LED unit (300).

The LED unit (300) comprises LED light sources (310) mounted in the toothbrush bristle body (210) for irradiating the LED light, and a substrate (320) for LED light source which is provided within the toothbrush bristle body (210) by the double injection molding of the toothbrush bristle body (210) and is electrically connected to the LED light sources (310).

Also, the connector modules (400′, 500′) comprise a first connector (400′) electrically connected to the substrate (320) for LED light source and a plug-type second connector (500′) for electrically connecting the first connector (400′) to the driving circuit substrate (140) connected with the driving power source (130).

The first connector (400′) consists of a plug-type connector (400′) mounted in series with the substrate (320) for LED light source. Such a plug-type connector (400′) comprises a plug (410′) defining the exterior and serving for alignment with the substrate (320) for LED light source, at least two strands of connection terminals (420′) protruding out of a front end portion of the plug (410′) from the inside of the front end portion and connected to the substrate (320) for LED light source, and a coupling part (430′) which is electrically connected to the connection terminals (420′) at a rear end portion of the plug (410′) and in which a second connector (500′) described later is accommodated and coupled.

One end portion of the plug (410′) adjacent to the substrate (320) for LED light source is formed in a trapezoidal shape, therefore, the connection terminals (420′) can be easily connected to the substrate (320) for LED light source at the time of assembly.

The second connector (500′) comprises a plastic material-made body part (510′) defining the exterior and pin parts (520′, 530′) formed long inside the body part (510′) and protruding from both end portions thereof, wherein such pin parts (520′, 530′) may comprise first pin parts (520′) protruding from one end portion of the body part (510′) and connected to the connection terminals (420′) of the first connector (400′), and second pin parts (530′) protruding from the other end portion of the body part (510′) and connected to the driving circuit substrate (140).

The first pin parts of the second connector are inserted between wall bodies formed above and below the coupling part of the first connector to be coupled with an insertion groove of the coupling part of the first connector, thus can be stably coupled.

In the case of such a electric toothbrush (10), a signal produced in the driving circuit substrate (140) is delivered to the substrate (320) for LED light source via the second pin parts (530′) and first pin parts (520′) of the second connector (500′) and then the coupling part (420′) and connection terminals (410′) of the first connector (400′), and the LED light sources (310)) mounted at the front end portion of toothbrush bristle body (210) can selectively emit the light

As described above, it can be appreciated that the electric toothbrush according to the examples of the present invention has a basic technical concept that stable driving control of the LED unit can be achieved through the connector modules interconnecting the driving power source and LED unit. Therefore, it is obvious to those having ordinary skill in the art that various modifications may be made within the scope of technical spirits of the present invention.

What is claimed:

1. An electric toothbrush comprising a handle having a vibrator and a toothbrush bristle assembly having a coupling space coupled with the vibrator,
the toothbrush bristle assembly comprises:
a toothbrush bristle body provided with a brush;
a LED unit mounted on the toothbrush bristle body for performing oral sterilization and tooth whitening treatment; and
a connector module having a first connector arranged within the toothbrush bristle body for interconnecting the LED unit and a driving power source and a second connector arranged within the vibrator.

2. The electric toothbrush according to claim 1, wherein the first connector comprises:
at least two strands of first connector pins;
a first connector socket formed at one end portion of the first connector pins; and
a first connector cover inserted and installed from the other end portion of the first connector pins, and
the second connector comprises at least two strands of pins inserted in the first connector socket.

3. The electric toothbrush according to claim 1, wherein the first connector comprises a plug-type connector mounted in series with a substrate for LED light source by at least two strands of pins, and
the second connector comprises a plastic material-made body part defining the exterior and at least two strands of pin parts formed long within the body part and inserted into the plug-type connector.

4. The electric toothbrush according to claim 1, wherein the toothbrush bristle body comprises a first toothbrush bristle body and a second toothbrush bristle body which are formed by a double injection molding method.

5. The electric toothbrush according to claim 1, wherein the toothbrush bristle assembly comprises:
a first toothbrush bristle body formed of synthetic resin by a first injection molding so that a first accommodation space is formed;
LED unit and first connector having a waterproof portion which has to avoid a contact with water or moisture and accommodated and joined in the first accommodation space of the first toothbrush bristle body; and
a second toothbrush bristle body formed by a second injection molding to be joined to the first toothbrush bristle body while accommodating the LED unit and the first connector in a second accommodation space so as to enclose the waterproof portion of the LED unit and first connector.

6. The electric toothbrush according to claim 1, wherein the LED unit comprises:
LED light sources mounted on the toothbrush bristle body for irradiating LED light; and
a substrate for LED light source which is provided within the toothbrush bristle body by the double injection molding of the toothbrush bristle body and controls the driving of LED light sources.

7. The electric toothbrush according to claim 6, wherein the LED light sources comprise red and white LED light sources.

8. The electric toothbrush according to claim 1, wherein the coupling space of the toothbrush bristle body and a shaft part of the vibrator inserted into the coupling space of the toothbrush bristle body are each provided in the form of dome.

9. The electric toothbrush according to claim 2, wherein the second connector has its outer circumferential surface formed corresponding to an inner circumferential surface of the vibrator so that the pins can be fixed within the vibrator without a shake.

10. A toothbrush bristle assembly comprising:
a toothbrush bristle body having a first toothbrush bristle body and a second toothbrush bristle body which are formed by a double injection molding method;
a LED unit comprising LED light sources for irradiating LED light to perform oral sterilization and tooth whitening treatment, and a substrate for LED light source mounted between the first toothbrush bristle body and the second toothbrush bristle body to control the driving of LED light sources; and
a connector module having a first connector arranged within the toothbrush bristle body for interconnecting the LED unit and a driving power source.
